(19) United States
(12) Patent Application Publication
(10) Pub. No.: US 2024/0091048 A1
(43) Pub. Date: Mar. 21, 2024

(54) CONNECTOR FOR AN OSTOMY COLLECTION DEVICE

(71) Applicant: Salts Healthcare Limited, Birmingham (GB)

(72) Inventors: Marcus Allen, Telford (GB); Mark Heynes, Newcastle-under-Lyme (GB)

(21) Appl. No.: 18/264,399

(22) PCT Filed: Feb. 3, 2022
(86) PCT No.: PCT/GB2022/050292

§ 371 Date: Aug. 4, 2023

(30) Foreign Application Priority Data
Feb. 5, 2021 (GB) 2101655.5

(51) Int. Cl. A61F 5/445; A61F 5/44

(52) U.S. Cl. CPC A61F 5/445; A61F 5/4405

(57) ABSTRACT

A connector (100) for an ostomy collection device (10), the ostomy collection device (10) having a housing (12) defining a collection volume for receiving and storing waste and a port (30) having an opening (32) which provides a fluid flow path to the collection volume, the connector (100) including: an engagement mechanism for engaging the port (30) of the ostomy collection device (10) including an engaging member (102), and an actuator (104) for moving the engaging member (102), wherein the engagement mechanism has a first state, in which the engaging member (102) is positioned to engage the port (30), and a second state, in which the engaging member (102) is positioned to disengage the port (30), wherein movement of the actuator (104) is generally transverse to the movement of the engaging member (102).

Embodiments of the present invention relate to a connector for an ostomy collection device.

Ostomy appliances are well known. Typically, a person having a stoma uses an ostomy appliance to collect waste from their stoma. The ostomy appliances collects a volume of waste and, once full or nearing full, the user either changes the appliance for a fresh one or empties the waste from the existing appliance.

This process works well while the user is able to visit a suitable space for changing/emptying their ostomy appliance—e.g. a bathroom or toilet. However, there may be times when the user does not wish to empty or change their appliance as often, for example, when travelling or while asleep.

In such times, it would be beneficial to provide an additional collecting volume that is connected to the existing ostomy appliance, so that the appliance does not need to be emptied as often. This functionality is currently provided by a flexible bag which is connected to the ostomy appliance.

Embodiments of the present invention seeks to alleviate one or more drawbacks of the prior art.

According to an aspect of the present invention we provide a connector for an ostomy collection device, the ostomy collection device having a housing defining a collection volume for receiving and storing waste and a port having an opening which provides a fluid flow path to the collection volume, the connector including:

an engagement mechanism for engaging the port of the ostomy collection device including
an engaging member, and
an actuator for moving the engaging member, wherein the engagement mechanism has a first state, in which the engaging member is positioned to engage the port, and a second state, in which the engaging member is positioned to disengage the port, wherein movement of the actuator is generally transverse to the movement of the engaging member.

According to a second aspect of the invention we provide an ostomy collection device for connecting to a connector according to the first aspect including:
a housing defining a collection volume for receiving and storing waste and a port having an opening which provides a fluid flow path to the collection volume,
the port including an engagement formation for connecting to the connector.

Further optional features according to the first and second aspects of the invention are provided in the appended claims.

Embodiments of the present invention are described, by way of example only, with reference to the accompanying drawings, in which:

FIG. 1 is a side view of an ostomy collection device,

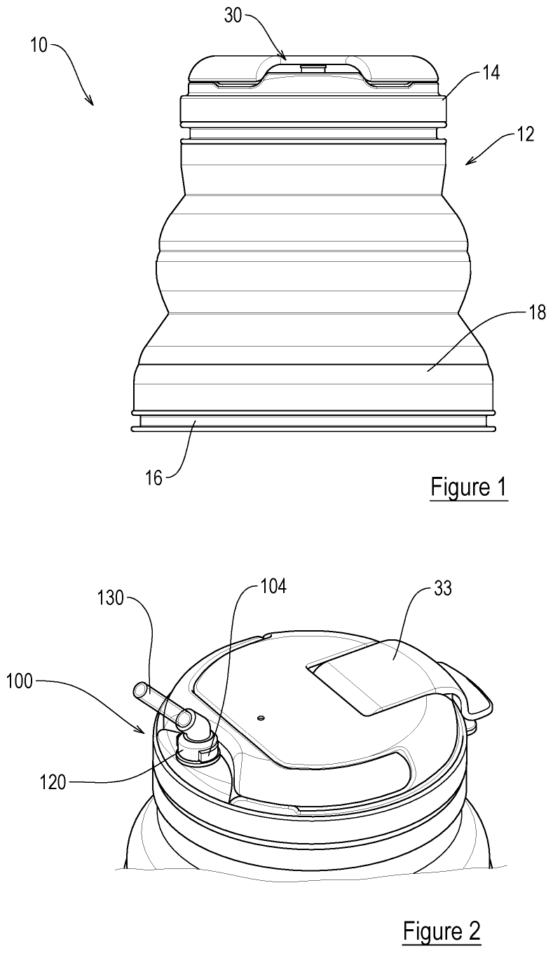

FIG. 2 is a perspective view of a top of an ostomy collection device and a connector,

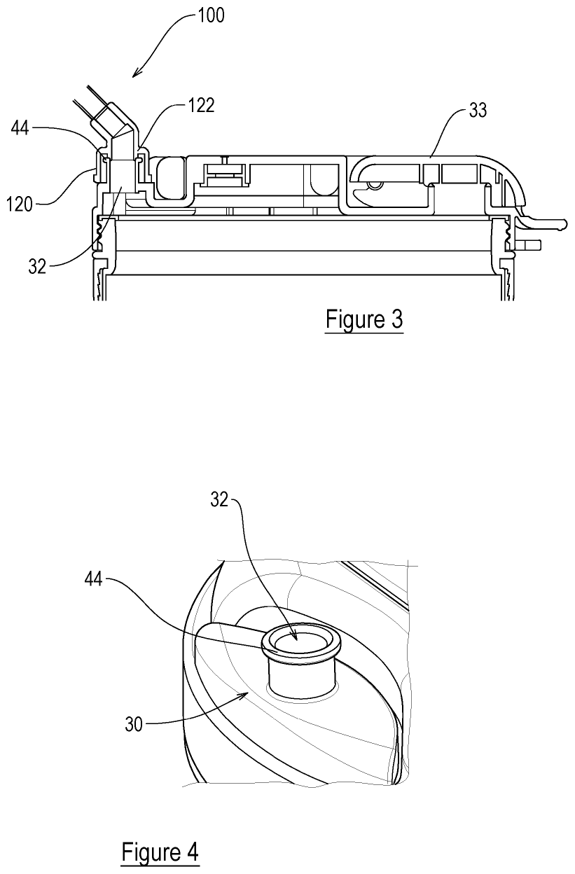

FIG. 3 is a cross-sectional view of a top of an ostomy collection device and a connector,

FIG. 4 is a perspective view of a part of a top of an ostomy collection device,

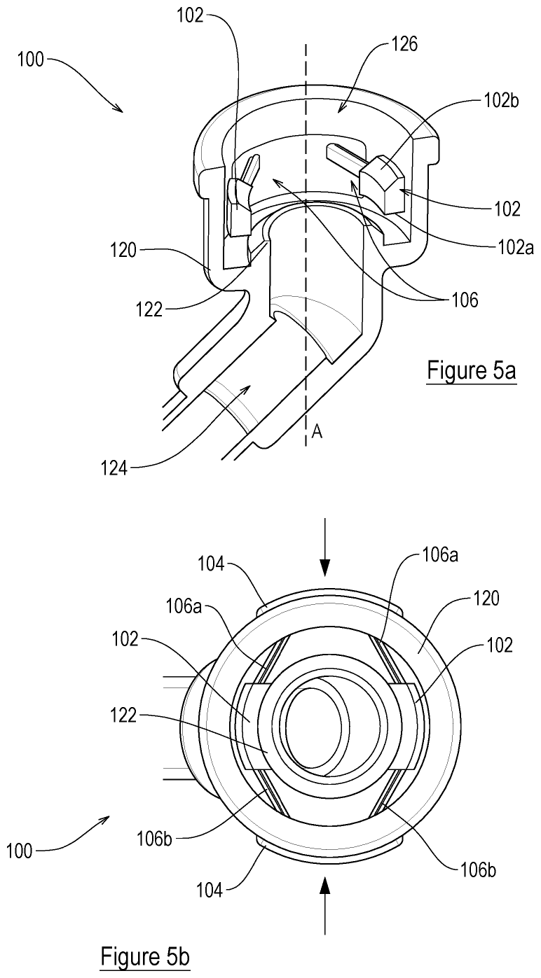

FIG. 5a shows a cross-sectional view of the connector, and

FIG. 5b shows a plan view of the connector.

An ostomy collection device 10 is shown in FIG. 1. The ostomy collection device 10 is configured to connect to an outlet or a tube connected to the outlet of a user's ostomy appliance that is connected about their stoma (and collecting waste exiting the stoma). The ostomy collection device 10 is particularly suited to urostomy use where the waste being collected is more fluid that other ostomy waste outputs and can, therefore, be emptied easily from the collection vessel into a toilet or waste disposal.

The ostomy collection device 10 includes a housing 12, which defines a collection volume for receiving and storing waste from an ostomy appliance. The ostomy collection device 10 also includes a port 30 (and, in some examples, an outlet for emptying the contents of the collection volume (i.e. to allow safe/clean waste disposal in a toilet or other appropriate receptacle).

In some embodiments (such as the example illustrated in FIG. 2), the housing 12 includes a lid portion 33 which covers the outlet and allows the outlet to be opened and closed, as desired by the user.

The port 30 includes an opening 32 and an engagement formation. The opening 32 provides a fluid flow path to the collection volume. The engagement formation is configured to connect to a connector 100 (discussed in more detail below). In some embodiments, the engagement formation of the port 30 includes a radially outwardly extending rim 44. In other words, the engagement formation include a portion that provides a ridge or flange that extends outwardly and provide a projection for an engagement member of the connector 100 to hold/attach to. In some embodiments, the rim 44 extends continuously around the circumference of the port 30 (but it should be appreciated that it may provide similar function if it is discontinuous/formed of multiple smaller individual projections about the port 30). In some embodiments, the engagement formation acts as a cam for an engaging member of the connector 100. In other words, a top surface of the engagement formation of the port 30 forms a slanted/angled surface which acts as a deflective surface. When an engaging member contacts the cam, it is deflected outwards along the angled surface and, thus, urged to a position in which the connector 100 can attach to the port 30.

The connector 100 which cooperates with embodiments of the ostomy collection device 10 will now be described in detail.

The connector 100 includes an engagement mechanism for engaging the port of the ostomy collection device 10. The connector 100 is configured to connect to the outlet of an ostomy appliance (not shown) either directly or via a tube or conduit 130. The connector 100 includes a housing 120, which provides an interface between the conduit/connection to the ostomy appliance and the ostomy collection device 10. The housing 120 includes an inlet 124 and outlet 126 connected by a fluid flow path which is configured to fluidly connect the connector 100 to the collection volume of the ostomy collection device 10.

In this present example, the housing 120 includes a skirt portion that extends over and around the port 30 when the connector 100 is connected to the ostomy collection device 10. The housing 120 also provides support for the parts of the engagement mechanism, so that the connector 100 can be connected and disconnected from the port 30.

The engagement mechanism includes an engaging member 102 and an actuator 104. The actuator 104 is configured to move the engaging member 102.

The actuator 104, in this example, projects through an opening in the housing 120 (to provide a portion that a user can interact with to alter the state of the engagement mechanism). In the illustrated example, a pair of actuators 104 is provided—each one projects through an opening in the housing 120 on opposing sides of the housing 120. Although the actuators 104 do not necessarily have to be on opposing sides of the housing 120, it may be advantageous to have this design because it is easier to grasp with by a user's finger and thumb if the actuators 104 are opposite one another.

Each actuator 104 is permitted to move along a single axis of movement. Since the actuators 104 are aligned with each other, this means that each actuator 104 moves towards and away from its counterpart (and towards and away from a central axis A—illustrated in FIG. 5a). The method of using the connector 100 is discussed in detail below.

The engaging member 102, in the present example, is inside the housing 120. The engaging member 102 includes a surface 102a that extends radially inwardly. In other words, the engaging member 102 includes a generally horizontal projection that provides a surface to contact the corresponding engagement formation of the port 30 and substantially prevent/inhibits the engaging member 102 from moving out of contact/disengaging from the port 30.

The engaging member 102 includes a cam 102b. The cam allows the engaging member 102 to deflect around a cam surface on the port 30. In the present example, the cam movement forces the engagement mechanism to move to its second state, when downward pressure is placed on the connector 100 (onto the top of the port 30).

In the illustrated example, the engagement mechanism includes a pair of engaging members 102. Each one of the pair of engaging members 102 is positioned on an opposing side of the housing 120 (and, thus, opposing sides of the port 30 when the connector 100 is attached).

The engagement mechanism has a first state and a second state. In the first state, the engaging member 102 is positioned to engage the port. In other words, when the engagement mechanism is in its first state, the connector 30 will connect/engage/lock onto the port 30. In the second state, the engaging member 102 is positioned to disengage the port. In other words, when the engagement mechanism is in its second state, the connector 30 will release from the port 30.

In order to move the engagement mechanism between its states (i.e. from the first state to the second state or vice versa), the actuator 104 moves generally transversely to the engaging member 102. In the illustrated examples which include a pair of actuators 104 and a pair of engaging members 102—both the actuators 104 move generally transversely to the movement of the engaging members 102.

The engagement mechanism permits rotation of the connector 100 relative to the ostomy collection device 10 (and the port 30) when in its first state and connected to the port 30. However, the connector 30 is not removable (cannot be lifted upwards and off the port 30) while the engagement mechanism is maintained in its first state and engaged on the port 30.

In some embodiments, radially inward motion of the actuator 104 causes radially outward motion of the engaging member 102. Likewise, radially outward motion of the actuator 104 causes radially inward motion of the engaging member 102.

In some embodiments, when the engagement mechanism is in its first state, the actuator 104 is in its radially outermost position and the engaging member 102 is in its radially innermost position. The first state is shown in FIG. 5b, which shows a plan view looking upwards into the connector 30 (the housing 120 extends around the outside). As can be seen, the actuators 104 project outwards of a housing 120 in their first state position (i.e. the position they are in when the engagement mechanism is in its first state). The engaging members 102 are spaced from the housing 120 wall (also in their first state position).

In some embodiments, the actuator 104 and the engaging member 102 are offset from one another around the axis A (e.g. the actuators 104 and engaging members 102 alternate). In other words, the first and second actuator 104 is positioned about 90 degrees each of the first and second engaging member 102 (circumferentially around the central axis A).

It should be appreciated that the actuators 104 and the engaging members 102 are positioned at different radial positions from the central axis A with respect to each other in one or both of the first and second states (but this does not necessarily need to be the case).

In some embodiments, when the engagement mechanism is in its second state, the actuator 104 is in its radially innermost position and engaging member 102 is in its radially outermost position. In other words, the “orientation” of the actuator 104 and the engaging member 102 swap over. The actuator 104 is closer to the central axis A and the engaging member 102 is further away from the axis A with respect to the first state. In some embodiments, the actuator 104 (or an outer surface of the actuator 104) may be in a position that is substantially aligned with the housing 120 (the outer surface of the housing 120) or the actuator is positioned radially inwards with respect to the housing 120. Whereas the engaging member 102 moves to an outer position (e.g. rather than being spaced from the housing 120 (discussed above), the engaging member 102 is adjacent and/or contacting the housing 120).

In some embodiments, the engagement mechanism includes a deflectable linkage 106, which extends between the actuator 104 and the engaging member 102. In the illustrated example, the engagement mechanism has a pair of deflectable linkages 106, each of which connects the engaging member 102 to opposing actuators 104. In more detail, in this example, each deflectable linkage 106 has a first part 106a and a second part 106b. The first part 106a extends between the first actuator 104 and the first engaging member 102 and the second part 106b extends between the engaging member 102 and the second actuator 104. The second deflectable linkage 106 is configured in an analogous manner between the pair of actuators 104 and the second engaging member 102.

In some embodiments, the engagement mechanism is biased to one of the first and second states. In other words, when there is no force or pressure on the actuator 104, both the actuator 104 and the engaging member 102 return to a default position. In the present example, the engagement mechanism returns to its first state (the state shown in FIG. 5b) when not subject to other outside force. This means that the engagement mechanism returns to and maintains a state in which the engaging member 102 is in a radially inwards position (and, thus, when the connector 100 is connected to the port 30—the engaging member 102 automatically maintains the position to hold/grip to the engagement formation of the port 30).

To move the engagement mechanism to its second state (from the first state), inward pressure is placed on the actuators 104 (for example, a thumb and finger may grasp the connector and apply the desired radially inward pressure to the actuators 104). The pressure moves the actuators 104 inwards towards a central axis that extends longitudinally through the connector (illustrated by line “A” in FIG. 3). The direction of pressure and of movement of the actuators 104 is illustrated by arrows in FIG. 5b.

In some embodiments, the connector 100 includes a sealing formation 122 which is configured to seal against the port 30 when the connector 100 is connected to the port 30. The sealing formation 122 is generally annular and corresponds to the shape of the opening of the port 30, so that the connector 100 provides a substantially sealed engagement with the ostomy collection device 10 when being used.

The method of using the connector 100 will now be described. When the connector 100 is being moved into connection with the port 30, there are two alternatives for how the engagement mechanism may operate to bring the connector 100 into a locked/secure engagement with the port 30.

Transverse pressure is applied to the actuators 104 (e.g. generally inward pressure), which causes the engaging members 102 to move outwards (i.e. away from the central axis A). In more detail, the deflectable linkages 106 are resilient and, when the actuators are pressed inwards, the deflectable linkages 106 bend to force the attached engaging members 104 outwards. Further, the two movement axes of the actuators 104 and the engaging members 102 are transverse (e.g. generally perpendicular) to each other.

This action moves the engaging members 104 out of the path of the engagement formation on the port 30 and allows the connector 30 to be moved downwards (over the port 30).

Once the engaging members 102 are past the engagement formation on the port 30, and the actuators 104 are released, the actuators 104 and the engaging members 102 move back to their default positions (which means the engaging members 102 move radially inwards). In other words, the deflectable linkages 106 are no longer under force caused by the pressure on the actuator and flex back to their original positions, thus pulling the engaging members 102 back to their original positions.

Therefore, the connector 100 cannot then be lifted off the port 30 because the engaging member 102 is trapped under the engagement formation of the port 30.

Another way of moving the connector 100 into a connected condition is to use the camming mechanism (if it is present as this is not essential to the operation/structure of the connector 100). As the connector 100 is moved down over the port, the cam 102b on the engaging member 102 contacts the cam surface of the port. As more downward pressure is applied, the two surfaces interact, to move the engaging member 102 outwards (as the cam 102b is deflected outwards by the cam surface). Once past the engagement formation of the port 30, the engaging members 102 will automatically move inwards (as discussed above).

In order to remove the connector 100 from the port 30, the following process is used. The actuators 104 are pressed inwards to move the engaging members 102 outwards. Once the engaging members 102 are moved outwards further than the radial extent of the engagement formation of the port 30, then the connector 100 is able to move upwards and out of contact with the port 30/ostomy collection device 10.

An advantage of the discussed connector 100 is that an easier way of connecting a conduit to the ostomy collection device 10 is provided (thus, and easier way of connecting an ostomy appliance to the collection device 10 is provided). This connector 100 also provides a secure and sealed connection with the inlet of the ostomy collection device 10 at the same time as providing a connection that rotates easily. This means there is a minimised chance of leakage while maximising the flexibility of the connection and reducing the chances of the ostomy collection device 10 falling over.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. A connector for an ostomy collection device, the ostomy collection device having a housing defining a collection volume for receiving and storing waste and a port having an opening which provides a fluid flow path to the collection volume, the connector including:
an engagement mechanism for engaging the port of the ostomy collection device including:
an engaging member, and
an actuator for moving the engaging member, wherein the engagement mechanism has a first state, in which the engaging member is positioned to engage the port, and a second state, in which the engaging member is positioned to disengage the port, wherein movement of the actuator is generally transverse to the movement of the engaging member.

2. The connector according to claim 1, wherein the engagement mechanism is configured to permit rotation of the connector relative to the ostomy collection device when in its first state.

3. The connector according to claim 1, wherein radially inward motion of the actuator causes radially outward motion of the engaging member.

4. The connector according to claim 3, wherein when the engagement mechanism is in its first state the actuator is in its radially outermost position and the engaging member is in its radially innermost position.

5. The connector according to claim 3, wherein when the engagement mechanism is in its second state the actuator is in its radially innermost position and the engaging member is in its radially outermost position.

6. The connector according to claim 3, wherein the engagement mechanism is biased towards one of the first and second states.

7. The connector according to claim 6, wherein the state to which the engagement mechanism is biased is the first state.

8. The connector according to claim 3, wherein the engagement mechanism includes a deflectable linkage between the actuator and the engaging member.

9. The connector according to claim 1, wherein the engaging member includes a surface that extends radially inwardly.

10. The connector according to claim 1, wherein the engaging member includes a cam.

11. The connector according to claim 1, wherein the engaging member is a first engaging member, and wherein the engagement mechanism further includes a second engaging member.

12. The connector according to claim 11, wherein the first and second engaging members are spaced on opposing sides of the port.

13. The connector according to claim 1, wherein the actuator is a first actuator, and wherein the engagement mechanism further includes a second actuator.

14. The connector according to claim 13, wherein the first and second actuators are spaced on opposing sides of the port.

15. The connector according to claim 1, which further includes a housing which extends around the outside of the engaging member(s).

16. The connector according to claim 15, wherein the housing includes an opening in which the actuator is positioned.

17. The connector according to claim 16, wherein when the engagement mechanism is in its first state, the actuator projects radially outwardly of the housing.

18. The connector according to claim 1, further including a sealing formation which is configured to seal against the port when the connector is connected to the port.

19. The connector according to claim 15, wherein the housing includes an inlet and an outlet connected by a fluid flow path which is configured to fluidly connect the connector to the collection volume of the ostomy collection device.

20. The connector according to claim 1, further including a conduit which is configured to attach to an outlet of an ostomy appliance.

21. An ostomy collection device for connecting to a connector according to claim 1, the ostomy collection device including:
a housing defining a collection volume for receiving and storing waste and a port having an opening which provides a fluid flow path to the collection volume,
the port including an engagement formation for connecting to the connector.

22. The ostomy collection device according to claim 21, wherein the engagement formation includes a radially outwardly extending rim.

23. The ostomy collection device according to claim 22, wherein the rim extends continuously around the circumference of the port.

24. The ostomy collection device according to claim 21, wherein the engagement formation acts as a cam for an engaging member of the connector.
